(19) United States
(12) Patent Application Publication
(10) Pub. No.: US 2007/0238991 A1
(43) Pub. Date: Oct. 11, 2007

(54) Ultrasound method and apparatus for characterizing and identifying biological tissues

(75) Inventors: Amar Amararene, Montreal (CA); Sylvain Jalbert, St-Lazarc (CA)

Correspondence Address: BROUILLETTE & PARTNERS, METCALFE TOWER, 1550 METCALFE STREET, SUITE 800, MONTREAL, QC, H3A-1X6, CA

(73) Assignee: JALTEC BIOMEDICAL INC.

(21) Appl. No.: 11/338,823

(22) Filed: Jan. 25, 2006

(51) Int. Cl. A61B 8/00

(52) U.S. Cl. 600/437; 600/442

(57) ABSTRACT

A method and apparatus are disclosed for characterizing and identifying tissues in a given body part using a transmitted ultrasound signal, wherein the method comprises receiving an ultrasound signal in response to the transmission of the transmitted ultrasound signal toward the given body part, computing an attenuation coefficient for the received ultrasound signal, the attenuation coefficient being indicative of the attenuation of the transmitted ultrasound signal in the tissue and identifying the tissue using the computed attenuation coefficient.

### CROSS-REFERENCE TO RELATED APPLICATIONS

This is the first application filed for the present invention.

### TECHNICAL FIELD

This invention relates to the field of ultrasound. More precisely, this invention pertains to a method and apparatus for characterizing and identifying biological tissues.

### BACKGROUND OF THE INVENTION

Various ultrasound apparatus are in use in medical diagnosis applications for humans and animals. These conventional ultrasound machines are based on the display of specular reflections from tissues. Tissue identification and clear distinction between normal and abnormal tissues is always left to a human appreciation. In vivo characterization of biological tissues using signal processing techniques on the reflected ultrasound signal is needed to make a firm decision concerning their identification. This is very important for medical diagnostic purposes but also for the animal industry. In the case of the animal industry, there is a dramatic need for pork producers for example to have an accurate estimation of the marbling (intramuscular fat) and for an early diagnosis of pregnancy in sows using ultrasound machines. At the present time, the prior art uses mainly image processing techniques to estimate the amount of marbling and the best portable echographs can only detect pregnancy with sufficient precision at day 21 after insemination. A more accurate estimation of the amount of marbling and an earlier pregnancy detection based on tissue characterization techniques may have a significant economic impact for pork producers. In general, there is an enormous need for animal producers to have a viable tool to follow the whole reproduction cycle from the oestrus and ovulation detection to the different steps of the embryo evolution. There is also a significant demand for measuring the amount of back fat.

### SUMMARY OF THE INVENTION

According to a first aspect of the invention, there is provided a method for characterizing and identifying a tissue in a given body part using a transmitted ultrasound signal, the method comprising receiving an ultrasound signal in response to the transmission of the transmitted ultrasound signal toward the given body part, computing an attenuation coefficient for the received ultrasound signal, the attenuation coefficient being indicative of the attenuation of the transmitted ultrasound signal in the tissue and identifying the tissue using the computed attenuation coefficient.

According to another aspect of the invention, there is provided an apparatus for characterizing and identifying tissue in a given body part, the apparatus comprising an ultrasound transceiver for transmitting an ultrasound signal to the given body part and receiving a corresponding RF signal from the given body part, an attenuation coefficient providing unit receiving the corresponding received RF signal, computing a corresponding attenuation coefficient for the received RF signal, the attenuation coefficient being indicative of the attenuation of the transmitted ultrasound signal in the tissue and a tissue identifying unit receiving the attenuation coefficient, detecting a corresponding tissue for the corresponding attenuation coefficient and providing a tissue indication signal.

According to another aspect of the invention, there is provided an imaging apparatus for displaying an image of a body part indicative of a tissue, the apparatus comprising an ultrasound transceiver for transmitting an ultrasound signal toward the given body part and receiving a corresponding RF signal from the given body part, an attenuation coefficient providing unit receiving the corresponding received RF signal, computing a corresponding attenuation coefficient for the received RF signal, the attenuation coefficient being indicative of the attenuation of the transmitted ultrasound signal in the tissue, a tissue identifying unit receiving the attenuation coefficient, detecting a corresponding tissue for the corresponding attenuation coefficient and providing a tissue indication signal and a display unit receiving the corresponding tissue and displaying the image of the body part indicative of the tissue.

According to another aspect of the invention, there is provided an imaging apparatus for displaying a parametric image of the ultrasound attenuation coefficient of a tissue, the apparatus comprising an ultrasound transceiver for transmitting an ultrasound signal toward the region of interest of the given tissue and receiving a corresponding RF signal from the region of interest of the given tissue, an attenuation coefficient providing unit receiving the corresponding received RF signal, computing a corresponding attenuation coefficient for the region of interest of the given tissue, the attenuation coefficient being indicative of the attenuation of the transmitted ultrasound signal in the region of interest of the given tissue, a display unit receiving the corresponding attenuation coefficient of the region of interest of the given tissue and displaying the image of the attenuation coefficient of the region of interest of the given tissue.

According to another aspect of the invention, there is provided an imaging apparatus for displaying a parametric image of the ultrasound attenuation coefficient of a tissue, the parametric image being displayed on a color scale, each color corresponding to a given identified type of tissue ; for example a blue color will be affected to a low attenuating tissue and a red color to a high attenuating tissue, while in between colors will be attributed to intermediate attenuating tissues.

According to another aspect of the invention, there is provided an apparatus for asserting a state of pregnancy of a mammal, the apparatus comprising an ultrasound transceiver for transmitting an ultrasound signal toward the womb of the mammal and receiving a corresponding RF signal, an attenuation coefficient providing unit receiving the corresponding received RF signal, computing a corresponding attenuation coefficient for the received RF signal, the attenuation coefficient being indicative of the attenuation of the transmitted ultrasound signal in the womb of the mammal, a tissue identifying unit receiving the attenuation coefficient and detecting a corresponding tissue for the corresponding attenuation coefficient and a state of pregnancy information providing unit, receiving the corresponding tissue and comparing the received corresponding tissue to a plurality of tissue signals each indicative of a state of pregnancy to provide the state of pregnancy of the mammal.

According to another aspect of the invention, there is provided a method for asserting a state of pregnancy of a mammal, the method comprising providing an RF signal received in response to an ultrasound signal transmitted in the womb of the mammal, computing an attenuation coefficient for the received RF signal, the attenuation coefficient being indicative of the attenuation of the transmitted ultrasound signal in the womb, identifying a tissue in the womb of the mammal using the computed attenuation coefficient and identifying the state of pregnancy of the mammal using the identified tissue.

### BRIEF DESCRIPTION OF THE DRAWINGS

Further features and advantages of the present invention will become apparent from the following detailed description, taken in combination with the appended drawings, in which:

FIG. 1 is a block diagram which shows an embodiment of an apparatus for identifying a tissue of a body part using a transmitted ultrasound signal; the apparatus comprises a processing unit, an attenuation coefficients providing unit, an ultrasound transducer, a memory unit and a display unit;

FIG. 2 is a block diagram which shows a first embodiment of an attenuation coefficients providing unit;

FIG. 3 is block diagram which shows a second embodiment of an attenuation coefficients providing unit wherein an adaptative time gain control is used;

FIG. 4 is a flowchart which shows how the apparatus for identifying a tissue of a body part operates according to one embodiment, according to a first step a received RF signal is provided, according to a second step an attenuation coefficient is computed for the received RF signal and according to a third step a corresponding tissue is identified using the computed attenuation coefficient;

FIG. 5 is a flowchart showing a first embodiment for providing the received RF signal;

FIG. 6 is a flowchart showing a second embodiment for providing the received RF signal, wherein a computed time gain control is used to compensate for the attenuation of the ultrasound transmitted signal when propagating in the tissue;

FIG. 7 is a flowchart showing an embodiment for computing the time gain control;

FIG. 8 is a flowchart showing how the computing of the attenuation coefficient is performed according to one embodiment;

FIG. 9 is a flowchart showing how a corresponding tissue is identified using the computed attenuation coefficient according to a first embodiment wherein an image comprising an identification of at least one tissue is displayed;

FIG. 10 is a flowchart showing how a corresponding tissue is identified using the computed attenuation coefficient according to another embodiment of the invention wherein a diagnostic is further provided; and

FIG. 11 is block diagram showing an embodiment of an apparatus for providing an indication of a state of pregnancy of a mammal.

It will be noted that throughout the appended drawings, like features are identified by like reference numerals.

### DETAILED DESCRIPTION OF AN EMBODIMENT

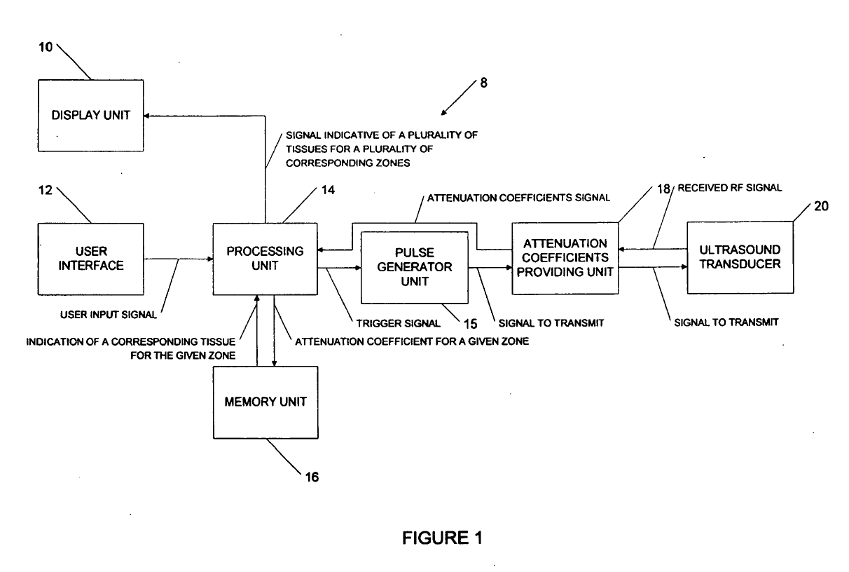

Now referring to FIG. 1, there is shown a first embodiment of an apparatus 8 for identifying a tissue using a transmitted ultrasound signal.

The apparatus 8 comprises a display unit 10, a user interface 12, a processing unit 14, a memory unit 16, an attenuation coefficients providing unit 18 and an ultrasound transducer 20. It will be appreciated that an attenuation coefficient is indicative of ultrasound energy losses by thermal dissipation, scattering and other forms of energy losses in a given tissue where an ultrasound wave is propagated.

The user interface 12 is used to enable an operator to provide a user input signal. The skilled addressee will appreciate that the user interface 12 may comprise at least one of a keyboard and a mouse. In an embodiment, the user interface 12 comprises an ON/OFF button, a command for a general menu with a predefined configuration, an input signal to set up the receiver amplifier gain (optional), a command to freeze the system, a zoom command (optional), a command to adjust the display unit 10.

The display unit 10 is used to display at least one image to the operator. It will be appreciated by the skilled addressee that the display unit 10 may be selected from a group consisting of Cathode Ray Tubes (CRT) screens, plasma screens, Liquid Crystal Display (LCD) screens, video glasses, projectors or the like. In an embodiment, the display unit 10 comprises one of an LCD screen and video glasses.

The processing unit 14 is used to process data as explained further below. The skilled addressee will appreciate that the processing unit 14 may be selected from a group consisting of dedicated processors, processors, field programmable gate area (FPGA) circuits, digital signal processing (DSP) circuits, microcontroller circuits, system on chip (SoC), or the like. In one embodiment, the processing unit 14 comprises one of microcontroller circuits and a system on chip (SoC).

The memory unit 16 is used to store data and is connected to the processing unit 14. The skilled addressee will appreciate that various type of memory may be used. It will be further appreciated that the amount of memory used depends on various factors known to the skilled addressee.

The attenuation coefficients providing unit 18 is used to compute the attenuation coefficient using a received RF signal. Its functionality will be discussed in more detail below.

The ultrasound transducer 20, also referred to as a probe, is used to convert electrical energy into ultrasound energy and vice versa, the transmit energy being propagated as an ultrasound wave in a body part. In one embodiment, the ultrasound transducer 20 may be a mechanical transducer or composed of a plurality of piezoelectric transducer elements forming a transducer array, the probe elements may range in number from 1 (i.e. single or monoelement transducer) to many elements, the maximum number of elements. It will be appreciated that the maximum number of elements is only restricted by technological limitations. The probe elements may be arranged to form specific arrays as for example a linear or phased array, in some applications curved arrays are also utilized as well as other array arrangements. The ultrasound transducer may be formed of a conventional material such as lead zirconate titanate (PZT) or may be an array of capacitive micromachined ultrasound transducers (CMUTs). In this embodiment, each probe element converts a high voltage electrical signal to an ultrasound signal transmitted through the body part. It will be appreciated that in one embodiment of the invention the frequency range of the ultrasound transducer 20 may be comprised between 0.2 MHz up to 50 MHz or higher, transducers with center frequencies such as 2, 3.5, 5, 7.5, 10 MHz, or higher may be used. The skilled addressee will appreciate that the ultrasound signal used may alternatively be a positive or negative rectangular pulse, a sine wave or any other form of excitation used in ultrasound machines. The pulse repetition frequency (PRF) of the transmitted signal is calculated to achieve the best trade off between an optimum image display and the best power management of the portable device.

More precisely and still referring to FIG. 1, the user interface 12 provides a user input signal to the processing unit 14 depending on the operator. In accordance with the user input signal provided by the user interface 12, the processing unit 14 provides a signal to trigger the pulse generator 15 which starts firing pulses with the desired PRF to the attenuation coefficients providing unit 18. The attenuation coefficients providing unit 18 generates a corresponding signal to transmit which is transmitted to the ultrasound transducer 20. The ultrasound transducer 20 provides an ultrasound signal to the body part and in response a received RF signal is provided by the ultrasound transducer 20 to the attenuation coefficients providing unit 18. The attenuation coefficients providing unit 18 analyzes the received ultrasound signal and provides an attenuation coefficients signal indicative of the attenuation of the transmitted ultrasound signal. The processing unit 14 receives the attenuation coefficients signal provided by the attenuation coefficient providing unit 18. The processing unit 14 provides an attenuation coefficient for a given zone to the memory unit 16.

An indication of a corresponding tissue for the attenuation coefficients for the given zone is received by the processing unit 14. The processing unit 14 then generates a signal indicative of a plurality of tissues for the plurality of corresponding zones and the processing unit 14 further provides the signal indicative of the plurality of tissues for a plurality of corresponding zones to the display unit 10.

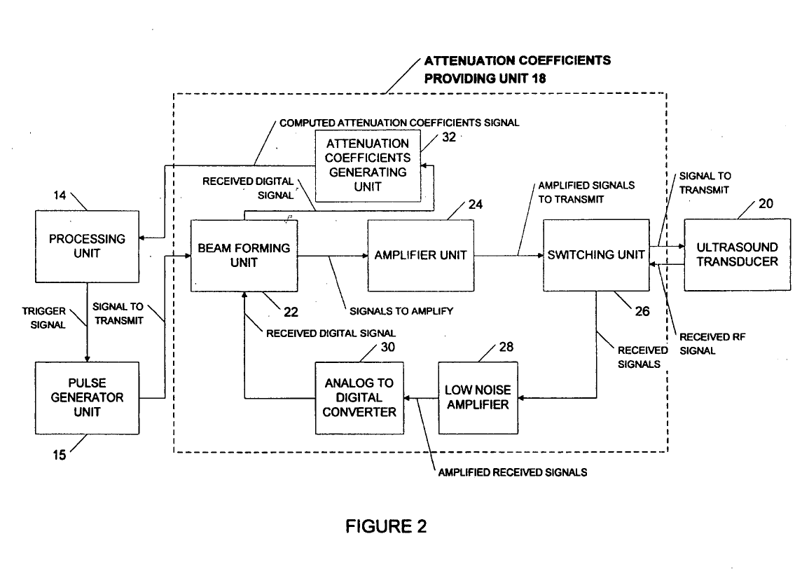

Now referring to FIG. 2, there is shown an embodiment of the attenuation coefficients providing unit 18 of the apparatus 8.

The attenuation coefficients providing unit 18 comprises a beamforming unit 22, an amplifier unit 24, a switching unit 26, a low noise amplifier (LNA) 28, an analog to digital converter 30 and an attenuation coefficients generating unit 32. The beamforming unit 22 is used, inter alia, to create signals with the appropriate delay times to produce the desired beam focusing and beam steering at the emission, the resulting signals are then amplified before being sent to the ultrasound transducer 20. The number of channels manipulated by the beamforming unit 22 is determined by the number of the probe elements to be activated to create the ultrasound beam. The beamforming unit 22 is further used at reception to reconstruct a received digital signal from the signals which are captured by the different elements of the probe unit 20. It will be appreciated that the beamforming unit 22 computes the appropriate time delays to reconstruct the signal to amplify.

The amplifier unit 24 is used to amplify signals intended to excite the different elements of the ultrasound transducer 20, these signals are received from the beamforming unit 22. In one embodiment, at the output of the amplifier unit 24, signals to transmit are negative high voltage pulses having a voltage located between -40 and -180 volts or higher. The signals to transmit to the ultrasound transducer 20 may also be a sine wave or all other signal shape with an appropriate voltage.

The switching unit 26 comprises several transmit/receive switches (T/R switches), it is used to separate received signals from signals to transmit to the ultrasound transducer 20.

It will be appreciated by the skilled addressee that the low noise amplifier (LNA) 28 is used to amplify the received signals issued from the different elements of the ultrasound transducer 20 and passing through the switching unit 26 to bring their amplitude to an appropriate level.

The analog to digital converter 30 is used to convert an analog signal to a digital signal.

In use, the processing unit 14 provides a signal to trigger the pulse generator unit 15 which transmits the transmit signal to the beamforming unit 22. The beamforming unit 22 generates corresponding signals to amplify which are provided to the amplifier unit 24. The amplifier unit 24 receives the signals to amplify provided by the beamforming unit 22 and amplifies the signals to provide amplified signals to transmit. The amplified signals to transmit are provided to the switching unit 26. The switching unit 26 receives the amplified signals to transmit provided by the amplifier unit 24 and provides signals to transmit to the ultrasound transducer 20.

Upon receiving a reflected ultrasound signal, the ultrasound transducer 20 provides received RF signals of its different elements to the switching unit 26. The switching unit 26 provides corresponding received signals to the low noise amplifier (LNA) 28. The low noise amplifier (LNA) 28 receives the received signals provided by the switching unit 26 and amplifies the received signals to provide amplified received signals. The amplified received signals are provided to the analog to digital converter 30. The analog to digital converter 30 receives amplified received signals and provides received digital signals.

The beamforming unit 22 receives the received digital signals provided by the analog to digital converter 30 and reconstructs a received digital signal using appropriate delay times. The beamforming unit 22 provides a received digital signal to the attenuation coefficients generating unit 32.

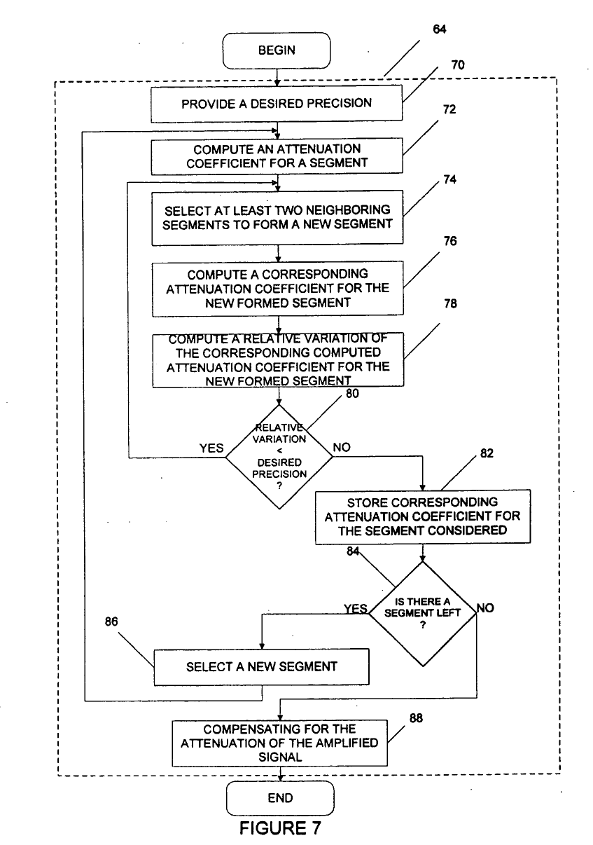

The attenuation coefficients generating unit 32 receives the received digital signal provided by the beamforming unit 22 and generates a plurality of attenuation coefficients. The generation of attenuation coefficients is further discussed below (see FIG. 7). The attenuation coefficient generating unit 32 further provides a computed attenuation coefficients signal to the processing unit 14.

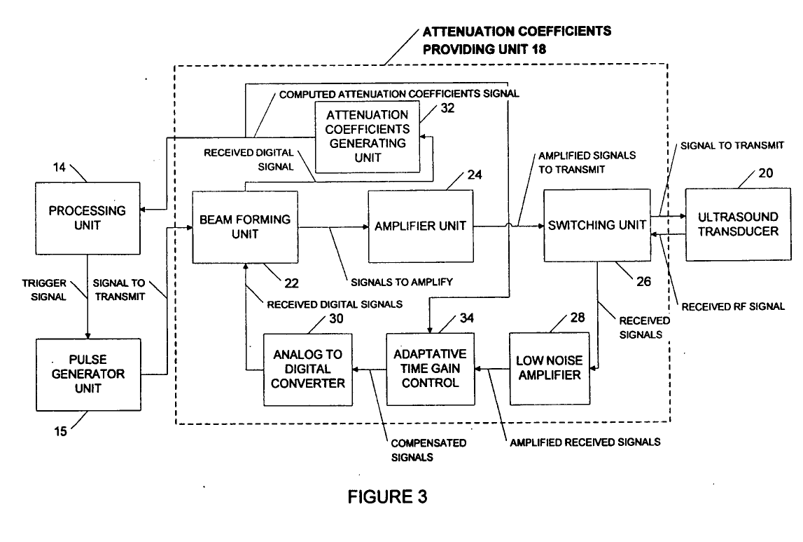

Now referring to FIG. 3, there is shown another embodiment of the attenuation coefficients providing unit 18. In this embodiment, the attenuation coefficients providing unit 18 further comprises an adaptative time gain control (ATGC) unit 34. The adaptative time gain control unit 34 is used to automatically compensate the attenuation of received signals provided by the low noise amplifier 28 and to provide corresponding amplified signals to the analog to digital converter 30.

The adaptative time gain control unit 34 operates using the computed attenuation coefficients signal provided by the attenuation coefficients generating units 32. In fact, it will be appreciated that the adaptative time gain control unit 34 amplifies the amplified received signal according to the type of tissue where the ultrasound signal is propagated. A received RF signal is divided into different segments, each of a them corresponding to a given tissue identified by the attenuation coefficient generating unit 32, and each segment will be amplified using the appropriate attenuation coefficient provided by the attenuation coefficients generating unit 32.

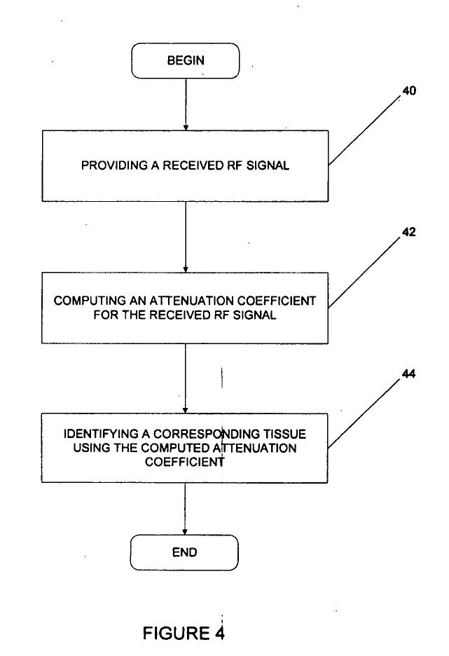

Now referring to FIG. 4, there is shown how the apparatus 8 for identifying a tissue of a body part operates according to one embodiment.

According to step 40, a received RF signal is provided. In one embodiment, the received RF signal is provided by the ultrasound transducer 20 to the attenuation coefficient providing unit 18.

According to step 42, an attenuation coefficient is computed using the received RF signal. In one embodiment, the attenuation coefficient for the received RF signal is computed using the attenuation coefficient providing unit 18. It will be appreciated that a plurality of attenuation coefficients may be computed using the received RF signal in the case where the received RF signal is received from a plurality of tissues.

According to step 44, a corresponding tissue is identified using the computed attenuation coefficient. In one embodiment, the corresponding tissue is identified using the computed attenuation coefficient using the processing unit 14 and the memory unit 16.

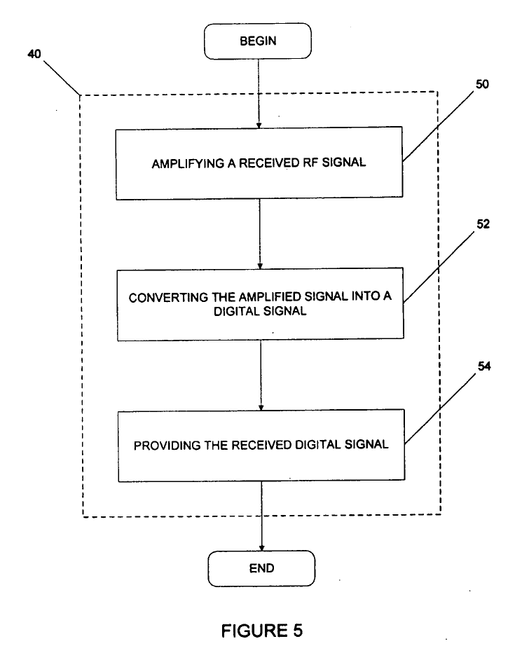

Now referring to FIG. 5, there is shown a first embodiment for providing the received RF signal.

According to step 50 a received RF signal is amplified. In one embodiment of the invention, the received RF signal is amplified using the low noise amplifier (LNA) 28.

According to step 52, the amplified signal is converted into a digital signal. In one embodiment, the amplified signal is converted into a digital signal using the analog to digital converter 30.

According to step 54, the received digital signal is provided. In one embodiment, the received digital signal is provided to the beamforming unit 22.

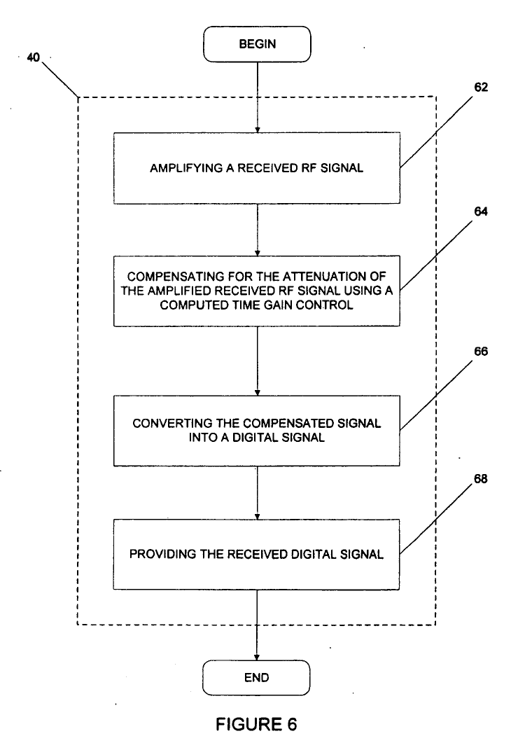

Now referring to FIG. 6, there is shown an alternative embodiment for providing the received ultrasound signal.

According to step 62, a received RF signal is amplified. In one embodiment of the invention, the received RF signal is amplified using the low noise amplifier (LNA) 28.

According to step 64, the amplified received RF signal is amplified again to compensate for attenuation of the transmitted ultrasound signal using a computed time gain control. In one embodiment of the invention, the amplified received RF signal is amplified using the adaptative time gain control unit 34.

According to step 66, the compensated signal is converted into a digital signal. In one embodiment of the invention, the amplified signal is converted into a digital signal using the analog to digital converter 30.

According to step 68, the received digital signal is provided. In one embodiment of the invention, the received digital signal is provided using the beamforming unit 22.

Now referring to FIG. 7, there is shown an embodiment for the amplification the amplified received RF signal using an adaptative time gain control.

According to step 70, a desired precision is provided. The skilled addressee will appreciate that various precision may be entered depending on an application sought and a type of tissue.

According to step 72, an attenuation coefficient is computed for a given segment.

According to step 74, at least two neighboring segments are selected to form a new segment.

According to step 76, a corresponding attenuation coefficient is computed for the new formed segment.

According to step 78, a relative variation of the corresponding computed attenuation coefficient is computed for the new formed segment.

According to step 80, a test is performed. More precisely, in the case where the computed relative variation is less than the provided desired precision, at least two other neighboring segments are selected to form a new segment according to step 74.

In the case where the computed relative variation is greater than or equal to the desired precision, and according to step 82, a corresponding attenuation coefficient for the segment considered is stored.

According to step 84, a test is then performed to find out if there is a segment left. In the case where there is at least one segment left and according to step 86, a new segment is selected among at least one of the segments left. In the case where there is no segment left, and according to step 88, an attenuation compensation of the amplified signal is performed using all computed attenuation coefficients.

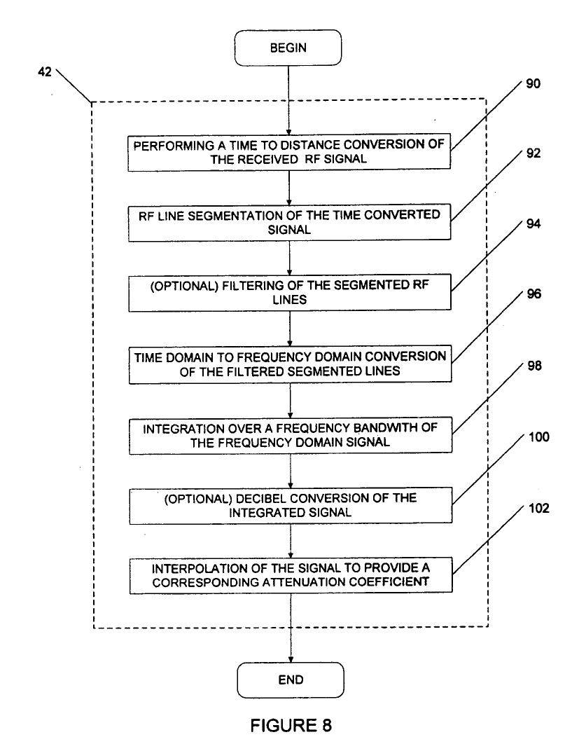

Now referring to FIG. 8, there is shown an embodiment for computing an attenuation coefficient for the received RF signal (step 42).

In one embodiment, the received RF signal comprises a two dimension matrix of RF data which are processed as explained herein below. Each column of the matrix of RF data or RF line represents an ultrasound signal as a function of time.

According to step 90, a time to distance conversion of the received ultrasound signal is performed. It will be appreciated that each RF line comprises a certain number of samples (N samples). The amplitude of a given sample number i is referred to a(i). The time separating two consecutive samples corresponds to the sampling period used. If u is the speed of sound and dt is the sampling period, then the corresponding distance of the sample number i is x(i)=i·dt·u.

According to step 92, an RF line segmentation of the previous converted signal is performed.

More precisely, each RF line is divided into a number of consecutive segments having a same size. Each segment must have a minimum number of samples. If the number of samples per segment is n, then the number of segments M is: M=N/n. While it is disclosed that each RF line is divided into a number of consecutive segments, the skilled addressee should appreciate that the RF line may alternatively be divided into a number of consecutive overlapping segments.

According to step 94, an optional filtering of the segmented RF line is performed.

It will be appreciated that the filtering may be applied to improve its spectrum. It will be appreciated that Hamming as well as Gaussian filtering functions may be used for that purpose.

According to step 96, a time domain to frequency domain conversion of the filtered segmented line is performed. In one embodiment, a Fast Fourier Transform (FFT) is performed to achieve such time domain to frequency domain conversion.

The skilled addressee will appreciate that the result is a frequency evolution of the amplitude of the backscattered signal for each segment of an RF line. It will be appreciated that proper frequencies are determined using the sampling frequency.

According to step 98, an integration over a given frequency bandwidth of the frequency domain signal is performed.

For a given segment number j, an integration over frequency bandwidth f₁, f₂ of the ultrasound transducer is made. As an example, the two frequencies f₁ and f₂ are relative to the ultrasound transducer bandwidth determined at −6 dB. The result of the integration is a unique value which will be affected to the center of the corresponding segment j.

$$A ( j ) = {\int }_{{f}_{1}}^{{f}_{2}} {A}_{j} ( f ) ⅆ f$$

According to step 100, an optional logarithmic (decibel units) conversion of the integrated signal is performed.
A_{dB}(j)=20 log₁₀(j)

According to step 102, an interpolation of the signal is performed to provide a corresponding attenuation coefficient for each line.

A_{dB} is represented versus x for all segments of a given line. A linear interpolation is made for the graph A_{dB} versus x. The obtained linear function is y_{dB}=αₜₑₘₚX+β. The value of the linear function y_{dB}=αx+β is used to find out the attenuation coefficient. More precisely, the latter is αₜₑₘₚ normalized with respect to the center frequency of the ultrasound transducer, i.e. (f₁+f₂)/2. Accordingly,

$$α = 2 \cdot \frac{{α}_{temp}}{{f}_{1} + {f}_{2}} .$$

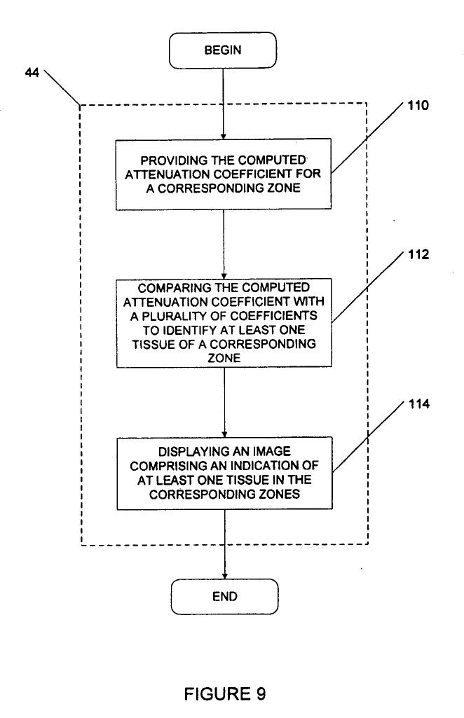

Now referring to FIG. 9, there is shown a first embodiment for identifying a corresponding tissue using the computed data attenuation coefficient (step 44).

According to step 110, the computed data attenuation coefficient is provided for a corresponding zone.

According to step 112, the computed data attenuation coefficient is compared with a plurality of coefficients to identify at least one tissue of the corresponding zone.

According to step 114, an image comprising an identification of at least one tissue in the corresponding zones is displayed.

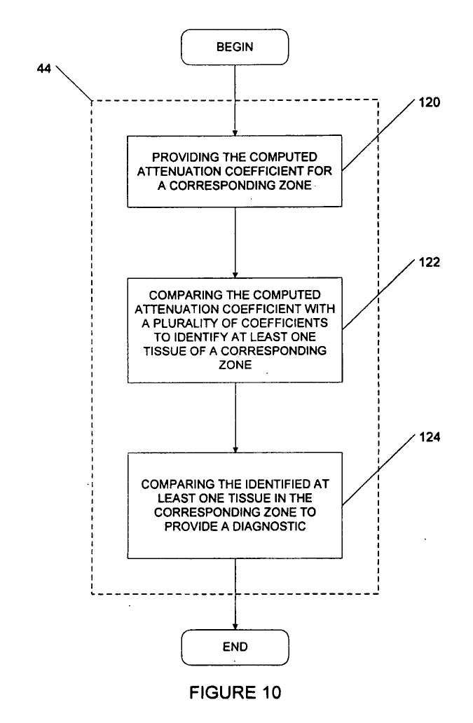

Now referring to FIG. 10, there is shown another embodiment for identifying a corresponding tissue using the computed attenuation coefficient (step 44).

According to step 120, the computed attenuation coefficient is provided for a corresponding zone.

According to step 122, the computed data attenuation coefficient is compared with a plurality of coefficients to identify at least one tissue of the corresponding zone.

According to step 124, the identified at least one tissue in the corresponding zone is compared to provide a diagnostic.

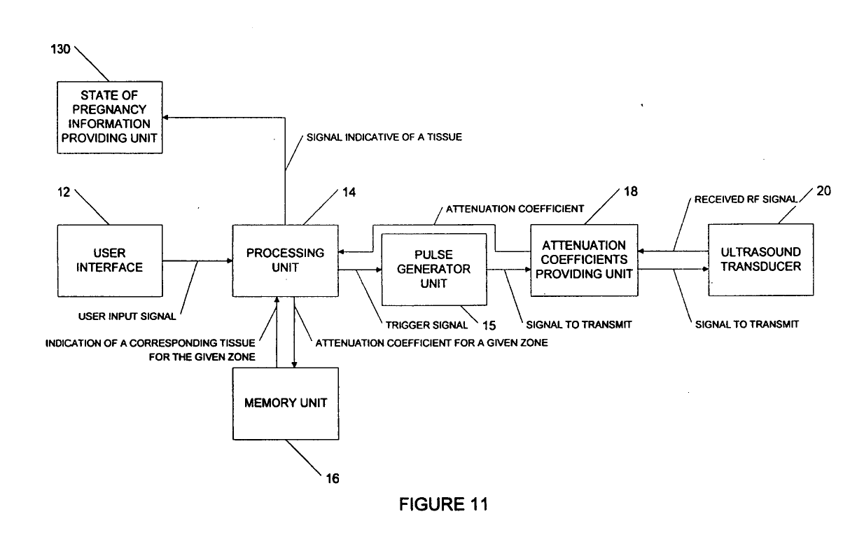

Now referring to FIG. 11, there is shown a further embodiment of an apparatus for providing a state of pregnancy information. The apparatus comprises the user interface 12, the processing unit 14, the memory unit 16, the attenuation coefficients providing unit 18, the ultrasound transducer 20 and the state of pregnancy information providing unit 130.

The user interface 12 provides the user input signal to the processing unit 14. The processing unit 14 provides a trigger signal to the pulse generator 15 which generates a signal to transmit to the attenuation coefficients providing unit 18. The attenuation coefficients providing unit 18 provides the signal to transmit to the ultrasound transducer 20. The ultrasound transducer 20 provides a received RF signal to the attenuation coefficient providing unit 18, the attenuation coefficient providing unit 18 provides a corresponding attenuation coefficient signal to the processing unit 14. The processing unit 14 provides at least one of the attenuation coefficient for a given zone to the memory unit 16 and receives from the memory unit 16 a corresponding indication of a tissue for the given zone.

According to the received indication of the corresponding tissue for the given zone, the processing unit 14 provides a signal indicative of an identified tissue. The signal indicative of a tissue is provided to the state of pregnancy information providing unit 130 which provides, preferably to an operator, a state of pregnancy depending on the tissue identified. It would be appreciated by the skilled addressee that such apparatus is of great advantage for detecting a state of pregnancy for mammals.

This is of great advantage for early pregnancy detection (i.e. prior to 18^{th} day). The skilled addressee will appreciate that a given attenuation in the womb as well the presence embryonic vesicles may be indicative of a pregnancy. In fact, it is known that sow pregnancy is accompanied by biological changes; for example simple folds are present in the wall of the sow's uterus during oestrus, after day five secondary and tertiary folds appear, other biological changes of the sow uterus are also observed during the gestation cycle (F. De Rensis et al, ‘Early diagnosis of pregnancy in sows by ultrasound evaluation of embryo development and uterine echotexture’, The Veterinary Record, September 2000). These biological changes may be advantageously detected using the method disclosed herein.

It will be appreciated by the skilled addressee that the apparatus and method disclosed herein enables a pregnancy detection in sow as early as the day 18 after insemination may be achieved. It will be further appreciated that the apparatus and method disclosed may also detect oestrus and ovulation in mammals and may follow the different steps of the embryo evolution. It may also provide a characterization and measurement of the amount of back fat and marbling.

It should also be appreciated that any type of tissues may be advantageously detected. For instance the method and apparatus disclosed may be used in the back fat and the marbling in the pork market. Malignancy tumors of mammals may also be detected.

The embodiments of the invention described above are intended to be exemplary only. The scope of the invention is therefore intended to be limited solely by the scope of the appended claims.

I claim:

1. A method for characterizing and identifying a tissue in a given body part using a transmitted ultrasound signal, said method comprising:
receiving an ultrasound signal in response to the transmission of said transmitted ultrasound signal toward said given body part;
computing an attenuation coefficient for said received ultrasound signal, said attenuation coefficient being indicative of the attenuation of the transmitted ultrasound signal in said tissue; and
identifying said tissue using the computed attenuation coefficient.

2. The method as claimed in claim 1, wherein said receiving of said ultrasound signal in response to the transmission of said transmitted ultrasound signal toward said given body part comprises amplifying a received analog ultrasound signal and converting said received analog ultrasound signal into a digital signal.

3. The method as claimed in claim 2, wherein said computing of said attenuation coefficient for said received ultrasound signal comprises performing a time conversion of said digital signal, performing a RF line segmentation of the time converted signal, performing a time domain to frequency domain conversion of said RF line segmented signal, performing an integration over a frequency bandwidth of said frequency domain converted signal and interpolating said integrated frequency domain converted signal to provide said computed attenuation coefficient.

4. The method as claimed in claim 3, wherein said RF line segmented signal is further filtered.

5. The method as claimed in claim 3, wherein said frequency domain converted signal is further converted into decibel.

6. The method as claimed in claim 1, wherein said identifying of said tissue comprises comparing the computed attenuation coefficient with a plurality of coefficients, each of said plurality of coefficients being indicative of a corresponding tissue to provide said identified tissue.

7. The method as claimed in claim 1, wherein said receiving of said ultrasound signal comprises amplifying a received analog ultrasound signal, filtering said amplified received analog ultrasound signal and converting said filtered signal into a digital signal.

8. The method as claimed in claim 7, wherein said filtering is performed using a computed time gain control.

9. The method as claimed in claim 7, wherein said computing of said attenuation coefficient for said received ultrasound signal comprises performing a time conversion of said digital signal, performing a RF line segmentation of said time converted signal, performing a time domain to frequency domain conversion of said RF line segmented signal, performing an integration over a frequency bandwidth of said frequency domain converted signal and interpolating said integrated frequency domain signal to provide said computed attenuation coefficient.

10. The method as claimed in claim 9, wherein said RF line segmented signal is further filtered.

11. The method as claimed in claim 9, wherein a decibel conversion is further performed on said frequency domain converted signal.

12. An apparatus for identifying a tissue in a given body part, said apparatus comprising:
an ultrasound transceiver for transmitting an ultrasound signal to said given body part and receiving a corresponding radio frequency (RF) signal from said given body part;
an attenuation coefficient providing unit receiving said corresponding received RF signal, computing a corresponding attenuation coefficient for said received RF signal, said attenuation coefficient being indicative of the attenuation of said transmitted ultrasound signal in said tissue; and
a tissue identifying unit receiving said attenuation coefficient, detecting a corresponding tissue for said corresponding attenuation coefficient and providing a tissue indication signal.

13. The apparatus as claimed in claim 12, wherein said attenuation coefficient providing unit comprises:
an analog to digital converter unit converting said received corresponding RF signal to provide a received digital signal; and
an attenuation coefficients generating unit receiving said received digital signal and providing said corresponding attenuation coefficient.

14. The apparatus as claimed in claim 13, further comprising:
a low noise amplifier receiving said received RF signal and filtering said received RF signal to provide a filtering RF signal to said analog to digital converter unit.

15. The apparatus as claimed in claim 14, further comprising:
a beamforming unit receiving a signal to transmit and providing a signal to amplify indicative of said ultrasound signal to transmit to said given body part; and
an amplifying unit receiving said signal to amplify and amplifying said signal to amplify to provide an amplified signal to said ultrasound transceiver.

16. The apparatus as claimed in claim 13, further comprising:
an adaptative time gain control unit receiving said received RF signal and performing an adaptative gain of said received RF signal to provide an amplified signal to said analog to digital converter unit.

17. An imaging apparatus for displaying an image of a body part indicative of a tissue, said apparatus comprising:
an ultrasound transceiver for transmitting an ultrasound signal toward said given body part and receiving a corresponding radio frequency (RF) signal from said given body part;
an attenuation coefficient providing unit receiving said corresponding received RF signal, computing a corresponding attenuation coefficient for said received RF signal, said attenuation coefficient being indicative of the attenuation of the transmitted ultrasound signal in said tissue;
a tissue identifying unit receiving said attenuation coefficient, detecting a corresponding tissue for said corresponding attenuation coefficient and providing a tissue indication signal; and
a display unit receiving said tissue indication signal and displaying said image of said body part indicative of said tissue.

18. The apparatus as claimed in claim 17, wherein said attenuation coefficient providing unit further comprises:
an analog to digital converter unit receiving and converting said received RF signal to provide a received digital signal; and
an attenuation coefficients generating unit receiving said received digital signal and providing a computed attenuation coefficients signal.

19. The apparatus as claimed in claim in claim 18, further comprising:
a low noise amplifier receiving said received RF signal and filtering said received RF signal to provide a filtered RF signal to said analog to digital converter unit.

20. The apparatus as claimed in claim 19 further comprising:
a beamforming unit receiving a signal to transmit and providing a signal to amplify indicative of said ultrasound signal to transmit to said given body part;
an amplifying unit receiving said signal to amplify and amplifying said signal to provide an amplified signal to said ultrasound transceiver.

21. The apparatus as claimed in claim 18, further comprising:
an adaptative time gain control unit receiving said received RF signal and performing an adaptative gain to provide an amplified signal to said analog to digital converter unit.

22. The apparatus as claimed in claim 17, wherein said display unit receives said corresponding tissue and displays said image of said body part indicative of said tissue using a plurality of colors, each color being indicative of a given tissue.

23. An apparatus for asserting a state of pregnancy of a mammal, said apparatus comprising:
an ultrasound transceiver for transmitting an ultrasound signal toward the womb of said mammal and receiving a corresponding radio frequency (RF) signal;
an attenuation coefficient providing unit receiving said corresponding received RF signal, computing a corresponding attenuation coefficient for said received RF signal, said attenuation coefficient being indicative of the attenuation of the transmitted ultrasound signal in the womb of said mammal;
a tissue identifying unit receiving said attenuation coefficient and detecting a corresponding tissue for said corresponding attenuation coefficient; and
a state of pregnancy information providing unit, receiving said corresponding tissue and comparing said received corresponding tissue to a plurality of tissue signals each indicative of a state of pregnancy to provide said state of pregnancy of said mammal.

24. The apparatus as claimed in claim 23, wherein said attenuation coefficient providing unit comprises:
an analog to digital converter unit receiving and converting said received RF signal to provide a received digital signal; and
an attenuation coefficients generating unit receiving said received digital signal and providing a computed attenuation coefficients signal.

25. The apparatus as claimed in claim in claim 23, further comprising:
a low noise amplifier receiving said received RF signal and filtering said received RF signal to provide a filtered RF signal to said analog to digital converter unit.

26. The apparatus as claimed in claim 25 further comprising:
a beamforming unit receiving a signal to transmit and providing a signal to amplify indicative of said ultrasound signal to transmit to said given body part;
an amplifying unit receiving said signal to amplify and amplifying said signal to provide an amplified signal to said ultrasound transceiver.

27. The apparatus as claimed in claim 24, further comprising:
an adaptative time gain control unit receiving said received RF signal and performing an adaptative gain to provide an amplified signal to said analog to digital converter unit.

28. The apparatus as claimed in claim 23, wherein said display unit receives said corresponding tissue and displays said image of said body part indicative of said tissue using a plurality of colors, each color being indicative of a given tissue.

29. A method for asserting a state of pregnancy of a mammal, said method comprising:
providing an ultrasound signal received in response to an ultrasound signal transmitted in the womb of said mammal;
computing an attenuation coefficient for said received ultrasound signal, said attenuation coefficient being indicative of the attenuation of the transmitted ultrasound signal in said womb;
identifying a tissue in said womb of said mammal using the computed attenuation coefficient; and
identifying said state of pregnancy of said mammal using said identified tissue.

30. The method as claimed in claim 29, wherein said identifying of said tissue in said womb of said mammal comprises comparing said computed attenuation coefficient with a plurality of coefficients, each indicative of a corresponding state of pregnancy of said mammal.

31. The apparatus as claimed in claim 17 wherein said display unit is selected from a group consisting of Cathode Ray Tubes screens, Liquid Crystal Display screens, video glasses and projectors.

32. The apparatus as claimed in claim 12, wherein said tissue identifying unit comprises:
a memory unit comprising for each of a plurality of attenuation coefficients an indication of a given tissue; and
a processing unit receiving said attenuation coefficient and accessing said memory unit using said attenuation coefficient to provide said tissue indication signal.

33. The apparatus as claimed in claim 32, wherein said processing unit is selected from a group consisting of processors, dedicated processors, field programmable gate area (FPGA) circuit, microcontroller circuits, system on chip (SOC).

34. The apparatus as claimed in claim 17, wherein said tissue identifying unit comprises:
a memory unit comprising for each of a plurality of attenuation coefficients an indication of a given tissue; and
a processing unit receiving said attenuation coefficient and accessing said memory unit using said attenuation coefficient to provide said tissue indication signal.

35. The apparatus as claimed in claim 34, wherein said processing unit is selected from a group consisting of processors, dedicated processors, field programmable gate area (FPGA) circuit, microcontroller circuits, system on chip (SOC).

36. The apparatus as claimed in claim 23, wherein said tissue identifying unit comprises:
a memory unit comprising for each of a plurality of attenuation coefficients an indication of a given tissue; and
a processing unit receiving said attenuation coefficient and accessing said memory unit using said attenuation coefficient to provide said detected corresponding tissue.

37. The apparatus as claimed in claim 36, wherein said processing unit is selected from a group consisting of processors, dedicated processors, field programmable gate area (FPGA) circuit, microcontroller circuits, system on chip (SOC).
